(19) United States
(12) Patent Application Publication
(10) Pub. No.: US 2017/0299538 A1
(43) Pub. Date: Oct. 19, 2017

(54) APPARATUS AND METHODS FOR PROVIDING DUAL-LAYER ENZYMATIC SENSOR

(71) Applicant: Verily Life Sciences LLC, Mountain View, CA (US)

(72) Inventors: Brian Marc Pepin, Oakland, CA (US); Angela Marie DiCiccio, Mountain View, CA (US); Jeffrey George Linhardt, Pleasanton, CA (US)

(73) Assignee: Verily Life Sciences LLC

(21) Appl. No.: 15/098,831

(22) Filed: Apr. 14, 2016

(51) Int. Cl. G01N 27/327; C12Q 1/00; B41J 2/01

(52) U.S. Cl. CPC G01N 27/3272; C12Q 1/003; B41J 2/01

(57) ABSTRACT

An enzymatic sensor and processes for making an enzymatic sensor are disclosed. In some implementations, a sensor is provided that includes a gel-enzyme layer for reacting with an analyte of interest to create an electrical signal corresponding to a concentration of the analyte in a sample. In addition, a cushion layer formed on the gel-enzyme layer to attenuate the effects of mechanical perturbations on the gel-enzyme layer and its concomitant distortion of a signal output of the sensor.

### BACKGROUND

The present disclosure generally relates to analyte sensors. More particularly, and without limitation, the present disclosure relates to enzymatic-based sensors for detecting analytes of interest.

Enzymatic-based sensors may be used to detect analytes of interest. With such sensors, enzymes are commonly immobilized in some type of matrix to render them more stable in solution. One known approach includes immobilizing an enzyme in a soft gel matrix, which while immobilizing the enzyme still allows diffusion of assay fluid into the matrix permitting the enzymes to come into contact with the analytes of interest. The interaction of the analytes of interest and the enzyme creates a signal that can be detected, and if desired, quantified. An example of this approach is the hydrogel matrix used for an active glucose-sensing contact lens to immobilize glucose oxidase as described in U.S. Patent Application Publication No. 2015/0173474, the contents of which are incorporated herein in their entirety.

One issue with soft gel matrices is that they can be deformed due to mechanical stress, and this deformation can change the matrix properties in nonlinear ways. These changes in the matrix properties can in turn affect the sensing output signal. Taking again the example of a glucose-sensing active contact lens, blinking of the eye can impart mechanical stress on the hydrogel matrix layer, which can compresses or distorts it partially or entirely. This changes the mechanical properties of the gel, which in turn can change the rate of diffusion of glucose through the gel. Because diffusion of glucose is proportional to the output signal, mechanical perturbations of the gel can directly affect the output signal.

Enzymatic sensors for various analytes including, e.g., glucose, have been proposed with multi-layer sensor structures, which can provide additional functionality and/or selectivity. However, these multi-layer structures are not designed to provide any type of mechanical isolation or mechanical noise reduction, largely because these sensors do not experience any mechanical stress during operation. For example, sensors inserted beneath the skin are not subject to regular mechanical stresses. This is a very different situation from, for example, an active contact lens, which is in a dynamic environment involving the constantly moving eyeball as well as mechanical force from the eyelids during blinking.

In view of the above and other factors, sensors with hydrogel-immobilized enzymes suffer from numerous drawbacks. These drawbacks are especially acute in situations where a sensor is contemplated as a wearable (e.g., for ongoing measurement of glucose in patients with diabetes mellitus) where a user's normal activity and motion can interfere with the measurement, and also in circumstances where a sensor cannot be protected from accidental exposure to mechanical perturbations.

### SUMMARY

The disclosed embodiments include sensors utilizing a soft gel matrix (e.g., a hydrogel) to immobilize and/or protect an enzymatic sensing component. The enzyme-gel matrix can be a cross-linked film. According to exemplary embodiments, the sensor is constructed on a substrate, and includes enzymes immobilized in a gel matrix deposited as a cross-linked film on top of electrodes. According to further aspects of the present disclosure, the enzyme could be glucose oxidase (GOx), the gel matrix could be a hydroxyethyl-methacrylate (HEMA) hydrogel, and the electrodes could be interdigitated platinum electrodes. In an illustrative embodiment, a glucose sensor where glucose diffuses into the hydrogel matrix to react with GOX creates H₂O₂, which diffuses to the platinum electrodes and oxidizes to create an amperometric signal that is proportional to the rate of diffusion of glucose. As disclosed herein, other illustrative embodiments are contemplated, which can employ different materials, different assay modalities, and/or different analytes, enzymes or electrochemical systems.

For these type of sensors employed in an active contact lens, for example, mechanical strain on the gel can directly affect the output signal. In an illustrative embodiment, the assay modality relies on amperometry, or the measure of current. Other approaches such as coulometry or potentiometry are also possible. Mechanical strain on the gel can alter the mechanical properties of the matrix, changing the rate of diffusion of glucose. When this rate of diffusion changes, the output changes. This is represented graphically in FIG. 1.

This disclosure proposes an exemplary embodiment of a sensor having a very soft “cushion” layer above the sensing gel layer (e.g., cross-linked film). In illustrative embodiments, the very soft cushion layer does not have any sensing ability, and is chosen to exhibit a high degree of permeability to the analytes of interest. During times of mechanical stress (e.g., compressive and/or shear) on the sensor gel stack, the cushion layer of the exemplary embodiment absorbs the majority of the strain, reducing stresses and strains on the sensing layer. As described herein, the softer the cushion layer is relative to the sensing layer, the more strain the cushion layer will absorb relative to the sensing layer.

Additional features and advantages of the disclosed embodiments will be set forth in part in the description that follows, and in part will be obvious from the description, or may be learned by practice of the disclosed embodiments. The features and advantages of the disclosed embodiments will be realized and attained by the elements and combinations particularly pointed out in the appended claims.

It is to be understood that both the foregoing general description and the following detailed description are examples and explanatory only and are not restrictive of the disclosed embodiments as claimed.

The accompanying drawings constitute a part of this specification. The drawings illustrate several embodiments of the present disclosure and, together with the description, serve to explain the principles of the disclosed embodiments as set forth in the accompanying claims.

### BRIEF DESCRIPTION OF THE DRAWINGS

FIG. 1 depicts schematically an example sensor device, consistent with embodiments of the present disclosure.

FIG. 2 depicts an example electrochemical assay system and application with an enzymatic-based sensor, consistent with embodiments of the present disclosure.

FIG. 3 depicts an exemplary process for forming enzyme sensors, consistent with embodiments of the present disclosure.

### DETAILED DESCRIPTION

The disclosed embodiments relate to sensors for detecting analytes using a hydrogel matrix and including at least one substantially permeable cushion layer to attenuate mechanical forces on the hydrogel matrix. As a result, the sensor device is more resistant to signal output distortions because of mechanical forces on the hydrogel matrix.

According to an example embodiment of the disclosure; an enzymatic sensor is disclosed having a substrate; a cross-linked film deposited over the substrate; at least one electrode communicatively coupled to the cross-linked film and fixedly coupled to the substrate; and a cushion layer deposited over the cross-linked film, wherein the cross-linked film comprises an enzyme chosen to catalyze an analyte of the enzymatic sensor, and further wherein the cushion layer is permeable and inert to the analyte.

According to another example embodiment of the disclosure, an enzymatic sensor comprising an analyte layer; and a cushion layer deposited over the analyte sensing layer, wherein the cushion layer has a Young's Modulus at least an order of magnitude lower than any other layer of the enzymatic sensor.

According to a further example embodiment, a method of reducing mechanical noise in an enzymatic sensor is disclosed, the method including providing an enzyme-gel matrix; and depositing a cushion layer over the enzyme-gel matrix; wherein the cushion layer is permeable to an analyte of the enzymatic sensor, and further wherein the cushion layer has a Young's Modulus less than the enzyme gel matrix.

According to a further example embodiment, a method for forming an enzyme sensor is disclosed having preparing a substrate surface to promote adhesion; depositing a gel-enzyme layer on the substrate; and depositing a gel cushion layer over the gel-enzyme layer.

In some aspects, an enzymatic-based sensor according to the present disclosure may be provided that includes a substrate; a gel matrix deposited over the substrate; at least one electrode communicatively coupled to the gel matrix and fixedly coupled to the substrate; and a cushion layer deposited over the gel matrix, wherein the gel matrix comprises an enzyme chosen to catalyze an analyte of the enzymatic sensor.

In some aspects, the cushion layer is permeable and inert to an analyte of interest. In some aspects, the cushion layer decreases the sensitivity of the enzymatic sensor to the analyte of interest by less than 10%. In further aspects, the cushion has a Young's Modulus less than the gel matrix. In some exemplary embodiments, the cushion layer has a Young's Modulus at least an order of magnitude lower than any other layer of the enzymatic sensor.

In some aspects, the enzymatic sensor includes a chemistry well, fixedly coupled to the substrate, and the gel matrix and cushion layer are disposed in a stack within the chemistry well.

According to further aspects of the disclosure, an axial force applied to the enzymatic sensor deforms the cushion layer and thereby attenuates forces from deforming the gel matrix.

According to a further aspect, a method of reducing mechanical noise in an enzymatic sensor is described, including depositing a cushion layer over an enzyme gel-matrix wherein the cushion layer is permeable to an analyte of the enzymatic sensor, and wherein the cushion layer has a Young's Modulus less than the enzyme gel matrix.

Reference will now be made in detail to embodiments of the present disclosure, examples of which are illustrated in the accompanying drawings. Where possible, the same reference numbers will be used throughout the drawings to refer to the same or like parts.

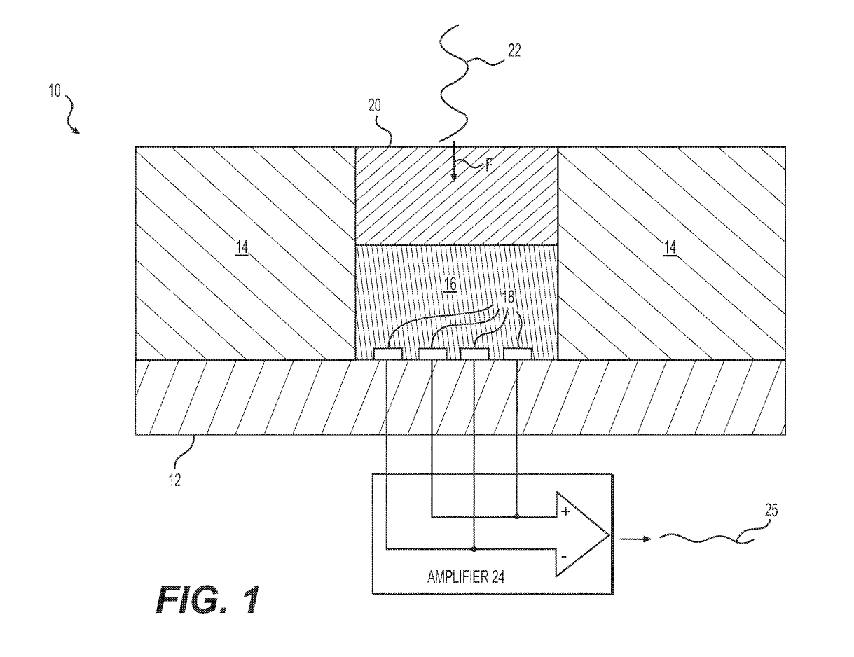

FIG. 1 schematically depicts, in cross-section, an example enzyme sensor device 10, consistent with embodiments of the present disclosure. As shown in FIG. 1, enzyme sensor 10 includes a substrate 12, a chemistry well 14, a cross-linked film 16, electrodes 18, and a cushion layer 20. The arrangement of these components in FIG. 1 is exemplary. Other arrangements are possible and will be appreciated from the present disclosure.

Substrate 12 is provided of a suitable material, which in exemplary embodiments is polymeric and which can be of a relatively rigid material, for example, polyethylene terephthalate (PET), polyolefin, polypropylene, silicon, or relatively resilient material such as polymethyl methacrylate (PMMA), polyhydroxyethylmethacrylate (polyHEMA), silicone hydrogels, or any combinations of materials. In illustrative embodiments embodied as an active contact lens, the material can include one or more biocompatible materials, such as those employed in contact lenses or other ophthalmic applications involving contact with the surface of the eye, for example the cornea, and can include hydrogels. For example, the substrate can be made of silicon with a silicon dioxide (SiO₂) passivation layer deposited thereon. In practice, the substrate can be any non-conductive material, such as glass, ceramics, or polymer layers (e.g., parylene, polyimide, PET).

Chemistry well 14 is formed on substrate 12 providing a volume for containing a cross-linked film 16. In illustrative embodiments, chemistry well 14 is formed by masking processes, such as positive mask (liftoff or screen-printing process), or a negative mask (plasma etching or a negative-tone, photodefinable polymer, such as SU-8). Chemistry well 14 may also be formed by direct-write laser ablation. Exemplary materials for chemistry well 14 include polymers (e.g., polyimide, parylene, SU-8, silicone). Deposition of the sensing layer (e.g., cross-linked film) can be simplified by selecting a well material that is significantly more hydrophobic than the substrate material. For example, the substrate can be SiO₂ and the well material can be silicone.

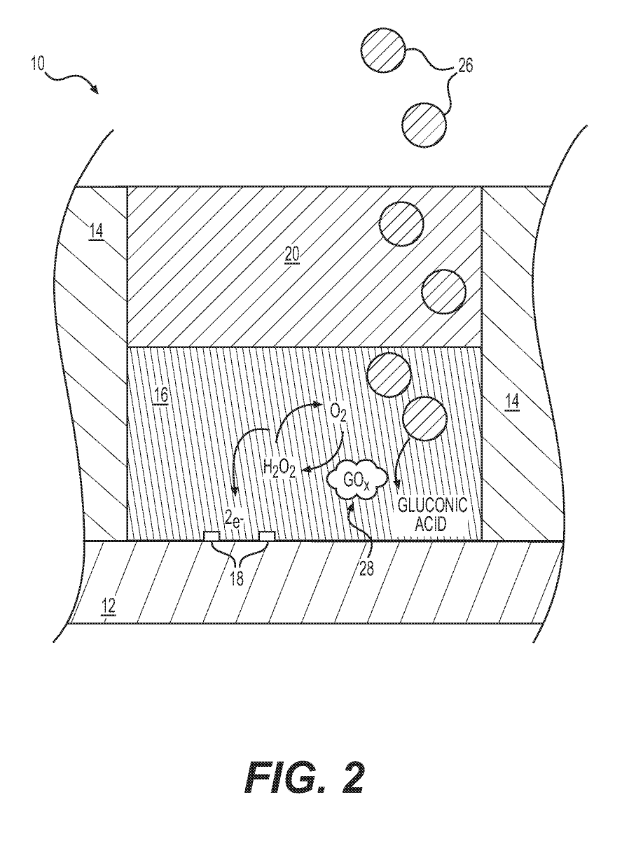

As shown in FIG. 1, electrodes 18 may be disposed at the bottom of chemistry well 14, but can be formed on other surfaces of the chemistry well or provided as extending or suspended features within the chemistry well. Electrodes 18 may be formed of a material suitable for performing electrochemical assays of a substance of interest (e.g., glucose) and based on the enzyme system employed (e.g., platinum electrodes for GOX). In exemplary embodiments intended for use in an active contact lens, the electrodes are formed of a biocompatible material. In an example discussed below with reference to FIG. 2, the electrodes of an amperometric electrochemical system using glucose oxidase are formed of platinum. This arrangement does not employ a mediator, but other systems can. Other electrode materials can include metals, semiconductors, nanoparticles, nano-metal oxides, quantum dots, etc. While four electrodes 18 are depicted in FIG. 1, any number of electrodes is possible, including combinations of anodes, cathodes, common and reference electrodes.

Cross-linked film 16 is disposed within chemistry well 14. Cross-linked film 16 can include an enzyme specific to a substance of interest dispersed in a hydrogel (e.g., a gel-enzyme matric). Cross-linking may be achieved by physical, covalent and/or associative means. Alternatively, the enzyme can be attached to the substrate 12 by an adhesion promoter, gel, physical or electrochemical adsorption, or the like. In the illustrative embodiment discussed with reference to FIG. 2, the enzyme is glucose oxidase (GOx), but other glucose-specific enzymes, as well as enzymes for other analytes, are possible. In an illustrative embodiment, the sensing chemistry can include GOx cross-linked with inactive proteins in an organic monomer film (e.g., (Hydroxyethyl)methacrylate (HEMA)), or a polymer film such as poly(HEMA), PVOH. In an illustrative embodiment, the polymer does not contain antioxidants or reducing agents. Hydrogels in illustrative embodiments include poly (ethylene glycol) acrylate (PEG), Bovine Serum Albumin (BSA), 2-acrylamidophenylboronic acid (2-APB), (3-acrylamidopropyl) trimethylammonium chloride (ATMA) and [2-(acryloyloxy)ethyl]-trimethylammonium chloride (AETA), and combinations thereof, but other hydrogels are possible. In an illustrative embodiment, the GOX enzyme can be cross-linked with BSA to form a film, which can be deposited on the sensor surface (e.g., substrate 12). In another illustrative embodiment, the GOX enzyme can be cross-linked into a HEMA gel, which forms a film that can be deposited on the sensor surface. A wide array of natural macromolecules can be used to cross-link an enzyme and polymer, for example, starches and other proteins like silk may be used.

In an illustrative embodiment, cross-linked film 16 is between about 1 to 10 microns thick, and cushion layer 20 is between 10 and 100 microns thick. The optimal thickness of cushion layer 20 is experimentally determined because gels do not exhibit linear behavior under dynamic loading conditions, and gels vary in their behavior. Equations can, however, be calculated to help optimize the thickness of cushion 20 based on design parameters such as dimensions, thickness of cross-linked film 16, the specific hydrogel used, and the contemplated dynamic perturbations to which the sensor will be exposed. In some embodiments, cushion 20 is the same thickness as cross-linked film 16. Because no material is 100% permeable, cushion 20 should not be so thick as to create degradation of the signal.

In illustrative embodiments, the enzymatic sensor cushion 20 is formed of a material having a Young's Modulus less than the gel-enzyme matrix. In some embodiments, the Young's Modulus of cushion 20 is an order of magnitude less than that of the gel-enzyme matrix 16. In an example, the cushion has a Young's Modulus ranging from about 0.3 to about 1.5 MPa. In another example, the cushion can withstand compressive and/or shear stress of about 2.6×10⁴ dynes/cm², which is the average force of an eye blink.

The chemical composition of the cushion 20 in an exemplary active contact lens will promote tear film lubrication and maintain a constant level of hydration representative of natural tear fluid.

Permeable cushion layer 20 is disposed in chemistry well 14 on cross-linked film 16. Alternatively, cushion layer 20 may be included as part of the cross-linked film 16. Cushion layer 20 forms an attenuator for mechanical perturbation 22 that translates to a force F on cushion 20. Cushion 20 absorbs and attenuates force F to protect cross-linked film 16 from mechanical perturbations 22. As depicted in the illustrative embodiment of FIG. 1, an amplifier 24 connected to electrodes 18 generates an output signal 26, whereby the oscillations resulting from mechanical perturbations 22 are greatly attenuated compared to an enzymatic sensor without cushion layer 20.

Cushion layer 20 has an adhesion strength to cross-linked film 16 able to withstand the shear force of a blink (e.g., greater than 2.6×10⁴ dynes/cm²). If cushion 20 undergoes deformation during the blink, there is advantageously little to no hysteresis to regain original conformation. Additionally the compressive strength of the cushion 20 is able to withstand failure under the pressure of the eyelid.

Cushion layer 20 is configured to be substantially permeable to the analyte of interest and to minimize the impact on the transit of the analyte to cross-linked film 16. In other words, cushion layer 20 is specifically designed and configured to avoid substantially modulating the amount of analyte reaching cross-linked film 16. Accordingly, cushion 20 is more permeable to the analyte of interest than cross-linked film 16 to avoid degrading the chemical signal. In an illustrative embodiment, cushion layer 20 is at least ten times more permeable than cross-linked film 16. Permeability can be measured by diffusion and/or electrochemistry. In another illustrative embodiment, the cushion 20 decreases the sensitivity of the enzyme sensor 10 to the analyte of interest by less than 10%, as compared to the same enzyme sensor with no cushion 20. Cushion layer 20 is also not specifically configured to filter molecules larger than the analyte of interest from reaching gel-enzyme matrix 16, although an infinitely permeable cushion is not theoretically achievable.

Cushion 20 can be made of suitable materials including hydrogels and polymers. In an illustrative embodiment, cushion 20 is made of a polyacrylamide. In an example, the cushion materials have water content between 30-90% without undergoing destructive swelling changes that damage morphology. Example materials include synthetic polymer gels used in the contact lens industry such as HEMA, PVOH, and silicones. Natural polymer gels include, for example, alginates, carageenans, silk, and protein. Hydrogels can also be characterized by their degree of cross-linking. In some embodiments, cushion 20 is cross-linked with other materials to provide for better adhesion to cross-linked film 16 and to chemistry well 14 when present. Examples include polysaccharides, such as alginate and chitosan.

In exemplary embodiments, the degree of cross-linking of the cushion hydrogels are such that cushion 20 is sufficiently permeable to the analyte of interest, such that the sensitivity of the enzyme sensor 10 to the analyte of interest is decreased by less than 10% when compared to the same enzyme sensor with no cushion 20. The crosslinking density and chemical composition of the cushion must be such that molecules of the analyte of interest, for example glucose, required for successful sensing chemistry, are able to freely diffuse through cushion 20 to access the gel-enzyme sensing layer 16. The composition can be tuned to block as many interfering molecules as possible either by size or charge exclusion principles. It should be noted that compositions that do not block interferents are not ruled out, as long as they allow passage of the glucose signal.

In illustrative embodiments, cushion layer 20 can be deposited using micro-scale deposition techniques, for example by jet forming or drop forming deposition methods. Exemplary techniques include positive displacement methods, such as nanoliter microinjection pipets, such as the NANOJECT available from Drummond Scientific. Other exemplary techniques include nanoliter non-contact dispensing, such as the PIPEJET available from BioFluidiX GmbH, and ink jet deposition.

Cushion 20 does not require an additional layer overlying it, but this disclosure is not limited to embodiments with only two layers in the stack. Additional layers could be added for any number of reasons, mechanical (e.g., load distribution or filtration), electrochemical (e.g., removal of interferents, microphages, etc.), aesthetic (including chromatic), etc. Additionally, illustrative embodiments (not shown) include those not utilizing chemistry well 14, where the sensor includes gel-enzyme layer 16 and cushion 20 without chemistry well 14.

As shown in FIG. 1, amplifier 24 can be provided to amplify the electrical signals gathered from electrodes 18. Oscillations in output signal 25, from the effects of mechanical perturbations 22 on gel-enzyme layer 16, are attenuated. Output signal 25 is further processed by a processor using conventional algorithms to convert output signal 25 into a glucose value.

Turning again to FIG. 2, an exemplary electrochemical assay system and application is shown embodied in enzyme sensor 10, consistent with embodiments of the present disclosure. Glucose molecules 26 pass through permeable cushion 20 and into cross-linked film 16, where they react with water and oxygen in the presence of GOx 28, creating gluconic acid and hydrogen peroxide (H₂O₂). The H₂O₂ is then catalytically reduced at platinum electrodes 18 resulting in the shedding of two electrons, which induces a current between a cathode-anode pair of electrodes 18. In an exemplary embodiment, the chemical composition of cushion 20 will be such that oxygen permeation is not limited and water content is greater than 30%, less than 90%. Upon uptake of water cushion 20 will not undergo changes in morphology (via swelling) that would compromise its ability to bind to chemistry well 14 or the cross-linked film 16.

Other electrochemical systems are possible, either with or without mediators, using amperometry, potentiometry or coulometry. Other enzymes can also be employed, either for the detection of glucose (e.g., glucose dehydrogenase) or other analytes.

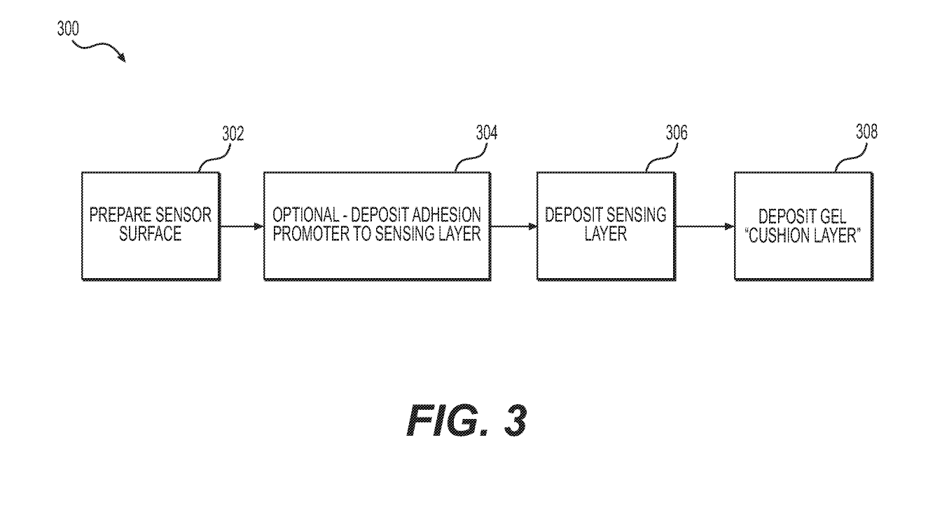

FIG. 3 depicts an illustrative process 300 for forming an enzyme sensor, consistent with embodiments of the present disclosure. At step 302, the sensor substrate surface is prepared, for example by isopropyl alcohol (IPA) rinse or O₂ plasma treatment, to clean the surface and promote adhesion. At optional step 304, an adhesion promoter is deposited on the substrate layer, for example a vapor phase treatment with 3-(Trimethoxysilyl)propyl methacrylate (A174 silane). At step 306, the cross-linked film (e.g., gel-enzyme layer or the sensing layer) is deposited, for example GOx/Bovine Serum Albumin (BSA) using inkjet deposition. At step 308, a gel cushion layer is deposited, for example, HEMA using a PIPEJET dispenser.

The foregoing description has been presented for purposes of illustration. It is not exhaustive and is not limited to precise forms or embodiments disclosed. Modifications and adaptations of the embodiments will be apparent from consideration of the specification and practice of the disclosed embodiments. For example, the described implementations include hardware and software, but systems and methods consistent with the present disclosure can be implemented as hardware alone. In addition, while certain components and arrangements have been described, other components and arrangements may be implemented, as will be appreciated from this disclosure.

Moreover, while illustrative embodiments have been described herein, the scope includes any and all embodiments having equivalent elements, modifications, omissions, combinations (e.g., of aspects across various embodiments), adaptations and/or alterations based on the present disclosure. The elements in the claims are to be interpreted broadly based on the language employed in the claims and not limited to examples described in the present specification or during the prosecution of the application, which examples are to be construed as nonexclusive. Further, the steps of the disclosed methods can be modified in any manner, including reordering steps and/or inserting or deleting steps.

The features and advantages of the disclosure are apparent from the detailed specification, and thus, it is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Words such as “and” or “or” mean “and/or” unless specifically directed otherwise. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

Other embodiments will be apparent from consideration of the specification and practice of the embodiments disclosed herein. It is intended that the specification and examples be considered as example only, with a true scope and spirit of the disclosed embodiments being indicated by the following claims.

What is claimed:

1. An enzymatic sensor comprising:
a substrate;
a cross-linked film deposited over the substrate;
at least one electrode coupled to the cross-linked film and coupled to the substrate; and
a cushion layer deposited over the cross-linked film,
wherein the cross-linked film comprises an enzyme chosen to catalyze an analyte, and
further wherein the cushion layer is permeable and inert to the analyte.

2. The enzymatic sensor of claim 1, further comprising a chemistry well, fixedly coupled to the substrate, and wherein the cross-linked film and cushion layer are deposited within the chemistry well.

3. The enzymatic sensor of claim 1, wherein an axial force applied to the enzymatic sensor deforms the cushion layer thereby preventing deformation of the cross-linked film.

4. The enzymatic sensor of claim 1, wherein the cushion layer is between about 10 microns and about 100 microns thick.

5. The enzymatic sensor of claim 1, wherein the cushion layer decreases the sensitivity of the enzymatic sensor to the analyte of interest by less than 10%.

6. The enzymatic sensor of claim 1, wherein the cushion layer is comprised of between about 30% and about 90% water.

7. The enzymatic sensor of claim 1, wherein the cushion layer includes at least one material selected from the group consisting of: polyacrylamide, HEMA, PVOH, silicones hydrogel, alginate, chitosan, carageenan, silk, and protein.

8. The enzymatic sensor of claim 1, wherein the cushion layer has a Young's Modulus less than the cross-linked film.

9. The enzymatic sensor of claim 1, wherein the cross-linked film comprises a gel-matrix of an enzyme and a polymer film.

10. A method of reducing mechanical noise in an enzymatic sensor, the method comprising:
providing an enzyme-gel matrix; and
depositing a cushion layer over the enzyme-gel matrix; wherein the cushion layer is permeable to an analyte of the enzymatic sensor, and
further wherein the cushion layer decreases the sensitivity of the enzymatic sensor to the analyte of interest by less than 10%.

11. An enzymatic sensor comprising:
a substrate;
a cross-linked film deposited over the substrate;
at least one electrode coupled to the cross-linked film and coupled to the substrate; and
a second layer deposited over the cross-linked film,
wherein the cross-linked film comprises an enzyme chosen to catalyze an analyte, the second layer is permeable and inert to the analyte, and an axial force applied to the enzymatic sensor deforms the second layer thereby preventing deformation of the cross-linked film.

12. The enzymatic sensor of claim 11, wherein the second layer decreases the sensitivity of the enzymatic sensor to the analyte by less than 10%.

13. The enzymatic sensor of claim 12, wherein the second layer has a Young's Modulus less than the gel matrix.

14. The enzymatic sensor of claim 11, wherein the second layer is between about 1 micron and about 10 microns thick.

15. The enzymatic sensor of claim 11, wherein the second layer is a cross-linked polymer and has a Young's Modulus at least an order of magnitude lower than any other layer of the enzymatic sensor.

16. The enzymatic sensor of claim 11, wherein the second layer is comprised of between about 30% and about 90% water.

17. The enzymatic sensor of claim 11, wherein the second layer includes at least one material selected from the group consisting of: polyacrylamide, HEMA, PVOH, silicones hydrogel, alginate, chitosan, carageenan, silk, and protein.

18. A method for forming an enzyme sensor, comprising:
preparing a substrate surface to promote adhesion;
depositing a gel-enzyme layer on the substrate; and
depositing a gel cushion layer over the gel-enzyme layer.

19. The method of claim 18, further comprising performing a isopropyl alcohol (IPA) rinse or O2 plasma treatment.

20. The method of claim 18, further comprising depositing an adhesion promoter on the substrate using a vapor phase treatment with 3-(Trimethoxysilyl)propyl methacrylate (A174 silane).

21. The method of step 18, wherein depositing the gel-enzyme layer comprises depositing GOx/Bovine Serum Albumin (BSA) using inkjet deposition.

22. The method of claim 18, wherein depositing the gel cushion layer comprises depositing HEMA using a PIPEJET dispenser.
